(19) United States
(12) Patent Application Publication
(10) Pub. No.: US 2007/0162017 A1
(43) Pub. Date: Jul. 12, 2007

(54) Method and apparatus for treatment of bones

(76) Inventor: Michael A. Gambale, Norwell, MA (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/600,983

(22) Filed: Nov. 16, 2006

Related U.S. Application Data
(60) Provisional application No. 60/740,100, filed on Nov. 28, 2005.

(51) Int. Cl. A61F 2/30

(52) U.S. Cl. 606/69

(57) ABSTRACT

A shank portion of a bone screw is moved through an opening in a bottom of a recess in a plate into engagement with bone in a patient's body. End portions of resiliently deflectable sections of a head portion of a bone screw are slid along the bottom of the recess during rotation of the bone screw. Resiliently deflectable sections of the head portion are deflected by engagement with the bottom of the recess. As the deflectable sections of the head portion are deflected, outer end surfaces on the resiliently deflectable sections of the head portion move into engagement with a side surface of the recess. The bone screw is retained against loosening by engagement of the outer end surfaces on the resiliently deflectable sections of the head portion with the side surface of the recess. An outer side surface of the bone screw is aligned with an outer side surface of the plate.

### RELATED APPLICATION

This application claims priority from U.S. Provisional Patent Application Ser. No. 60/740,100, filed Nov. 28, 2005, the subject matter of which is incorporated herein by reference.

### BACKGROUND OF THE INVENTION

The present invention relates to a new and improved method and apparatus for treating bone and more specifically to a plate which is secured to bone by screws.

During the treatment of bones in a patient's body, one or more metal plates have been secured to the bone by the use of screws. Although the metal plates may be utilized in the treatment of many different bones in a patient's body, they have advantageously been used in the past in association with neurosurgical and craniofacial operations. The plates have been secured to the bone by the use of screws and positioned beneath a patient's skin. Head portions of the screws have projected from the plates and caused objectionable bumps in the patient's skin. Known ways in which plates may be utilized in the treatment of bone are disclosed in U.S. Pat. Nos. 5,201,737; 5,413,577; 5,766,176; 6,692,498; and 6,942,665.

### SUMMARY OF THE INVENTION

The present invention relates to a new and improved method and apparatus for use in association with bone in a patient's body. The apparatus includes a plate which is secured to bone in the patient's body by an improved bone screw. When they are installed in the patient's body, the plate and the bone screw cooperate to retain the bone screw against loosening. The installed bone screw has an outer side surface which is aligned with an outer side surface of the plate to minimize projections from the plate.

The present invention has a plurality of different features. These features may be utilized together in the manner disclosed herein. Alternatively, the various features of the invention may be utilized separately and/or in combination with the features from the prior art.

### BRIEF DESCRIPTION OF THE DRAWINGS

The foregoing and other features of the invention will become more apparent upon consideration of the following description taken in connection with the accompanying drawings wherein:

FIG. 1 is a schematic illustration of bone on which plates of various types have been mounted for purposes of illustrating the invention;

FIG. 2 is an enlarged schematic sectional view depicting the manner in which an improved bone screw cooperates with any one of the plates of FIG. 1 during mounting of the plate on the bone in a patient's body;

FIG. 3 is a plan view, taken generally along the line 3-3 of FIG. 2, further illustrating the manner in which the bone screw cooperates with a plate;

FIG. 4 is a schematic illustration, generally similar to FIG. 3, depicting the manner in which a driver cooperates with the improved bone screw of FIGS. 2 and 3 to rotate the bone screw relative to the plate;

FIG. 5 is a schematic sectional view, generally similar to FIG. 2, illustrating a relationship between the improved bone screw and the plate after the bone screw has been installed in a bone in a patient's body, and FIG. 6 is a plan view, taken along the line 6-6 of FIG. 5, illustrating the manner in which the installed bone screw and plate cooperate to retain the bone screw against rotation relative to the plate.

### DESCRIPTION OF SPECIFIC PREFERRED EMBODIMENTS OF THE INVENTION

Bone 10 disposed in a patient's body has been illustrated schematically in FIG. 1. Although the bone 10 illustrated in FIG. 1 is a skull, it should be understood that the skull is representative of many bones in a patient's body and that the present invention may be utilized in association with bones other than skull bones.

Various types of plates 12, 14, 16 and 18 have been illustrated schematically in association with the bone 10. The plates 12-18 are representative of many different known types of plates which may be utilized. The plate 12 is a formable mesh and may have a construction similar to the construction in U.S. Pat. No. 5,766,176. The disclosure in the aforementioned U.S. Pat. No. 5,766,176 is hereby incorporated in its entirety by this reference thereto.

The plate 14 has a plurality of slots and vanes and is constructed in the general manner disclosed in U.S. Pat. No. 5,201,737. The plate 16 is an angle plate which is precontoured to have a configuration corresponding to the configuration of a portion of a bone on which the plate is to be installed. The plate 16 has the same general construction as is disclosed in U.S. Pat. No. 5,413,577. Plate 18 is a generic representation of a plate and has a generally rectangular configuration.

The plates 12-18 may be utilized in cranial and facial surgery during the performance of neurosurgical, plastic and reconstruction operations. Although the plates are advantageously utilized in maxillofacial and cranial surgery, they can be utilized for many different types of surgery in association with many different types of bones.

The plates 12-18 are formed of metal, specifically, titanium. However, it is contemplated that the plates 12-18 may be constructed of other materials, such as stainless steel or cobalt chrome molybdenum. If desired, the plates 12-18 may be formed of a bioabsorbable material. The plates 12-18 are relatively thin so as to minimize the extent to which the plates protrude from the bone 10.

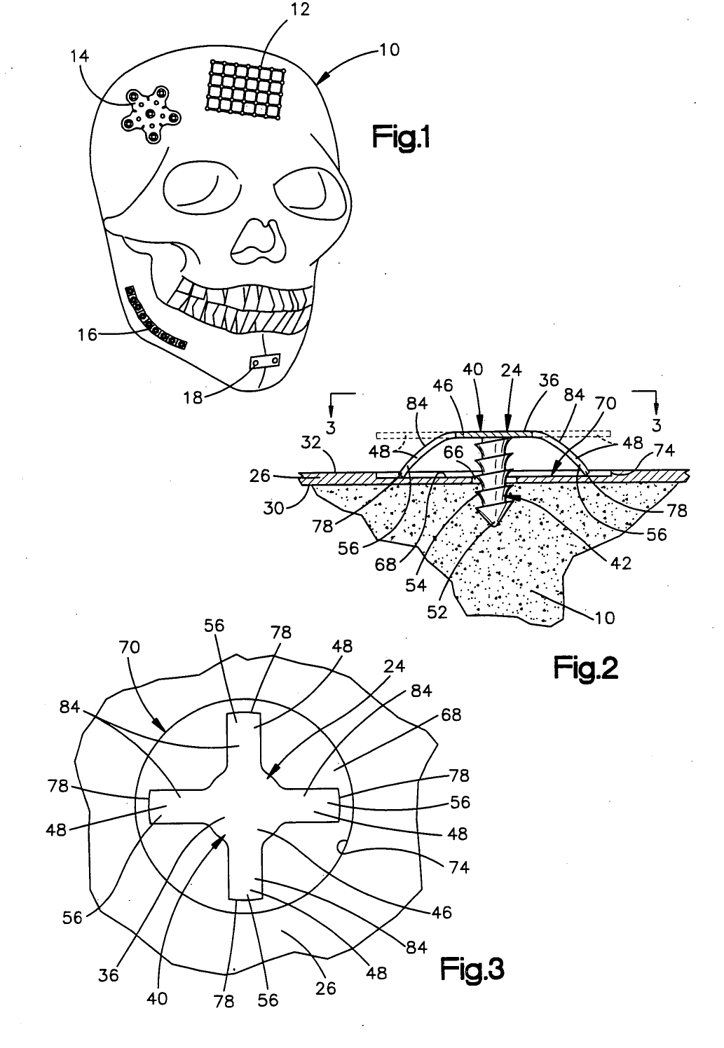

An improved bone screw 24 is illustrated in FIGS. 2 and 3 in association with a plate 26. The plate 26 may have a construction similar to the construction of any one of the plates 12-18. However, in the embodiment of the invention illustrated in FIG. 2, the plate 26 is a formable mesh having a construction similar to the construction of the plate 12. The plate 26 has an inner major side surface 30 which engages the bone 10 and an outer major side surface 32 which faces away from the bone. Although only one bone screw 24 is illustrated in FIGS. 2 and 3, it should be understood that there are a plurality of identical bone screws in the plate.

The bone screw 24 is movable between an initial condition of FIGS. 2 and 3 and an installed condition of FIGS. 5 and 6. When the bone screw is in the installed condition (FIG. 5), an outer side surface 36 of the bone screw is aligned with an outer major side surface 32 of a plate 36. This minimizes formation of a projection in a patient's skin which overlies the installed bone screw 24 and plate 26. When the bone screw 24 is installed in the manner illustrated in FIGS. 5 and 6, a head portion 40 of the bone screw 24 is engaged by the plate 26 to prevent loosening of the bone screw in the bone 10.

When the head portion 40 of the bone screw 24 is in the initial condition of FIG. 2, the head portion extends radially outward from and axially along the shank portion 42. The head portion 40 of the bone screw 24 includes a central portion 46 and a plurality of resiliently deflectable sections or arms 48. Although the central portion 46 may have a different configuration and the illustrated embodiment of the bone screw 24, the central portion 46 has a circular configuration.

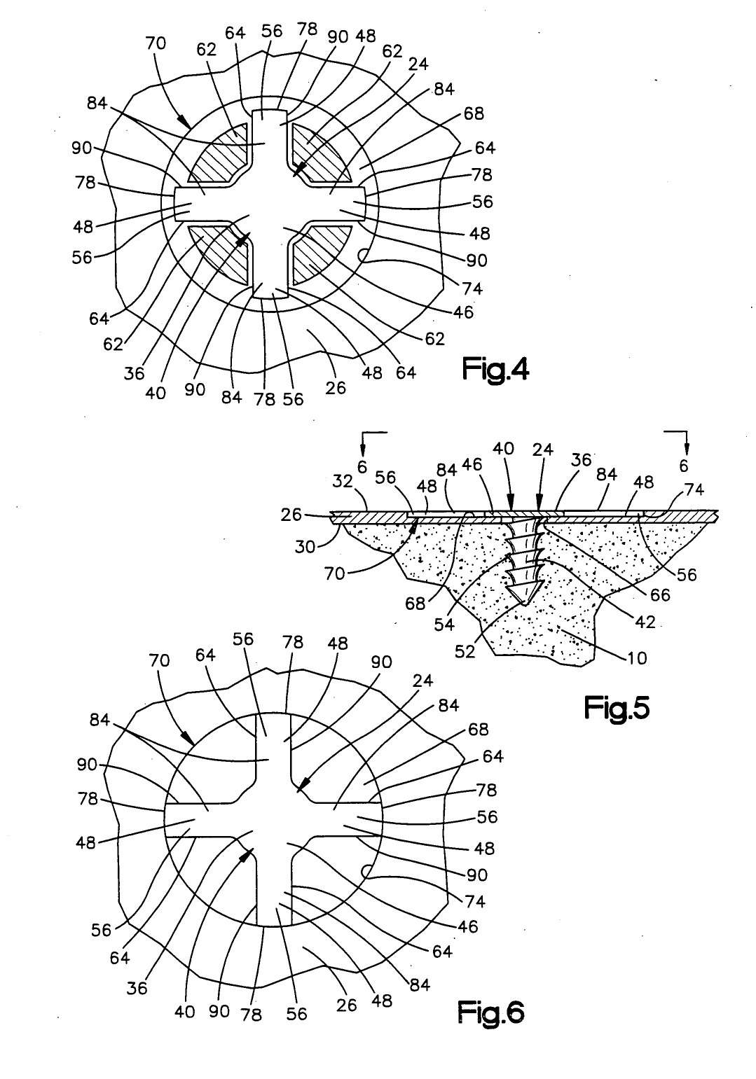

The resiliently deflectable sections or arms 48 extend generally radially outward from the central portion 46. In addition, the resiliently deflectable sections or arms 48 extend from the central portion 46 of the head portion 40 in a direction toward a leading end 52 (FIG. 2) of the shank portion 42. When the bone screw 24 is in the initial condition of FIG. 2, the resiliently deflectable sections or arms 48 are engaged by a driver 60 (FIG. 4). Rotation of the driver 60 about its central axis causes segments 62 of the driver to apply force against drive surfaces 64 on the resiliently deflectable sections are arms 48 to rotate the bone screw 24 in a clockwise direction (as viewed in FIG. 4).

At this time, the shank portion 42 of the bone screw 24 extends through a circular opening 66 at the center of a circular bottom surface 68 of a circular recess 70 in the plate 26. As the bone screw 24 continues to be rotated by the driver 60, the external thread convolution 54 on the shank portion 42 causes the shank portion to move downward (as viewed in FIG. 2) into the bone 10.

As the shank portion 42 begins to move into the bone 10, the end portions 56 on the resiliently deflectable sections or arms 48 move downward (as viewed in FIG. 2) toward the bottom surface 68 of the recess. As the shank portion continues to be rotated, radially inner corners on the end portions 56 move into engagement with the bottom surface 68 of the recess 70, in the manner illustrated schematically in FIG. 2. As the bone screw 24 continues to be rotated about its longitudinal central axis, the interaction between the external thread convolution 54 and the bone 10 causes the shank portion 42 to move downward (as viewed in FIG. 2) into the bone 10. As this time, the end portions 56 of the resiliently deflectable sections or arms 48 slide along the bottom surface 68 of the circular recess 70 at a location spaced from a cylindrical side surface 74 of the recess (FIGS. 2 and 3). The end portions 56 of the resiliently deflectable sections or arms 48 move along a circular path along the bottoms surface 68 of the recess 70. At the same time, force transmitted from the shank portion 42 to the head portion 40 presses the end portions 56 of the resiliently deflectable sections or arms 48 against the bottom surface 68 of the recess 70 with sufficient force to deflect the end portions of the arms radially outward toward the cylindrical side surface 74 of the recess 70.

As this occurs, the resiliently deflectable sections or arms 48 are deflected from their arcuately curving initial configuration (FIG. 2) toward a straight radially outwardly extending configuration illustrated dashed lines in FIG. 2. Thus, as the end portions 56 of the resiliently deflectable sections or arms 48 slide in a circular path along the bottom surface 68 of the recess 70, they also slide along the bottom surface in a direction away from the center of the recess toward the side surface 74 of the recess.

As the shank portion 42 of the bone screw 24 moves into the bone 10, arcuate end surfaces 78 on the resiliently deflectable sections or arms 48 move toward the side surface 74 of the recess 70 (FIG. 3). As the bone screw 24 approaches the installed condition of FIG. 5, the arcuate end surfaces 78 on the resiliently deflectable sections or arms 48 move into engagement with the cylindrical side surface 74 of the recess 70. Continued tightening rotation of the bone screw 24 into the bone 10 causes the arcuate end surfaces 78 on the resiliently deflectable sections or arms 48 to slide along the side surface 74 of the recess and to be pressed firmly against the side surface of the recess.

When the bone screw 24 reaches the installed condition of FIG. 5, the arcuate end surfaces 78 on the resiliently deflectable sections or arms 48 have been jammed against the side surface 74 of the recess with a wedging action which resists rotation of the bone screw in the tightening direction. At this time, there will be an interference fit between the arcuate end surfaces 78 on the resiliently deflectable arm sections 48 and the side surface 74 of the recess 70. This interference fit will block further rotation of the bone screw 24 by the driver 60 in a clockwise direction (as viewed in FIG. 4). Once this has occurred, the driver 60 is disengaged from the bone screw 24.

The interference fit between the arcuate end surfaces 78 on the resiliently deflectable sections or arms 48 and the side surface 74 of the recess 70 will retain the bone screw 24 against counterclockwise or loosening rotation relative to the plate 26. Therefore, the bone screw 24 is securely held in the installed condition of FIGS. 5 and 6 under the influence of force transmitted between the outer end surfaces 78 on the resiliently deflectable sections or arms 48 and the side surface 74 of the recess 70. This results in the bone screw being locked to the plate 26 so that the bone screw can not subsequently loosen.

When the bone screw 24 is in the initial condition of FIG. 2, arcuately curving outer side surfaces 84 on the resiliently deflectable sections or arms 48 extend transverse to the bottom surface 68 of the recess 70. As the bone screw 24 is rotated and tightened by the driver 60, the outer side surfaces 84 of the resiliently deflectable arm sections 48 move from the arcuately curving configuration shown in solid lines in FIG. 2 toward a linear configuration shown in dashed lines in FIG. 2. When the bone screw 24 is in the installed condition of FIG. 5, the outer side surfaces of the resiliently deflectable sections or arms 48 are linear and extend parallel to the bottom surface 68 of the recess 70. At this time, inner side surfaces on the resiliently deflectable sections or arms 48 are in flat abutting engagement with the bottom surface 68 of the recess 70.

The recess 70 has a depth which is the same as the thickness of the resiliently deflectable arm sections 48. Therefore, as the resiliently deflectable sections or arm 48 are deflected from the arcuately curving configuration of FIG. 2 toward the linear configuration of FIG. 5, the outer side surfaces 84 on the resiliently deflectable arm sections 48 move into alignment with the outer major side surface 32 of the plate 26.

In the embodiment of the invention illustrated in FIGS. 2-6, the cylindrical side surface 74 of the recess 70 has an axial extent which is the same as the thickness of the resiliently deflectable sections or arms 48 of the head portion 40 of the bone screw 20. Therefore, the outer side surfaces 84 of the resiliently deflectable sections or arms are disposed in a coplanar relationship with the major outer side surface 32 of the plate 26. However, it is contemplated that the cylindrical side surface 74 of the recess 70 may have an axial extent which is either slightly greater than or less than the thickness of the resiliently deflectable sections or arms 48 of the head portion 40 of the bone screw 24.

If the axial extent of the side surface 74 of the recess 70 is slightly greater than the thickness of the resiliently deflectable sections or arms 48, the outer side surfaces 84 of the resiliently deflectable arms will be offset downward (as viewed in FIG. 5) from the outer major side surface 32 of the plate 26. Similarly, if the resiliently deflectable sections or arms 48 have a thickness which is slightly greater than the axial extent of the side surface 74 of the recess 70, the outer side surfaces 84 of the resiliently deflectable sections or arms will be offset upwardly (as viewed in FIG. 5) from the outer side surface 32 of the plate 26. It is contemplated that, in order to minimize irregularities in the portion of a patient's skin which overlies the plate 26 and bone screw 24 that it would be desired to have the outer side surfaces 84 of the resiliently deflectable sections or arms 48 in substantial alignment with the outer major side surface 32 of the plate 26.

It should be understood that the bone screw 24 is shown larger than its actual size in FIGS. 2-6. When the bone screw 24 is used in association with cranial and/or facial osteosynthesis, the bone screw may have a length of approximately 4 mm and be utilized with a plate 26 which may have a thickness which is less than 1.0 mm. Of course, when the screw 24 is to be utilized in association with surgery other than maxillofacial and/or cranial surgery, the bone screw 24 and plate 26 may have dimensions other than the specific dimensions previously set forth herein. It is also contemplated that when a screw 24 and plate 26 are used for maxillofacial and/or cranial surgery, they may have dimensions which are smaller than or larger than the specific dimensions set forth herein. The specific dimensions with which the bone screw 24 and plate 26 are constructed will depend upon the environment in which they are to be utilized.

In the embodiment of the invention illustrated in FIGS. 1-6, the bone screw 24 is provided with four resiliently deflectable sections or arms 48 which extend outward from the central portion 46 of the head portion 40 of the bone screw. However, it is contemplated that either a greater or lesser number of resiliently deflectable sections or arms 48 may be provided if desired. For example, a pair of resiliently deflectable sections or arms 48 may be provided. When a pair of resiliently deflectable sections or arms 48 are utilized, each of the resiliently deflectable sections or arms may have an arcuate end surface 78 which extends for a relatively large distance along the cylindrical inner side surface 74 of the recess. The arcuate end surfaces 78 on the two resiliently deflectable sections or arms 48 may extend for approximately 90 degrees along the side surface 74 of the recess 70. Alternatively, the bone screw 24 may be constructed with a head portion 40 having a greater number of resiliently deflectable arm sections 48. For example, the head portion 40 of the bone screw 24 may be constructed with six resiliently deflectable sections or arms 48.

It is contemplated that the bone screw 24 may be constructed with resilient deflectable sections or arms 48 having end surfaces 78 which are disposed in a circular array which extends completely around the shank portion 42 of the bone screw 24 when the bone screw is in the initial condition of FIG. 2. If the bone screw 24 has such a construction, slits may be provided between the resiliently deflectable sections or arms 48 to facilitate axial deflection and radial expansion of the head portion 40 when the bone screw 24 is moved from the initial condition of FIG. 2 to the installed condition of FIG. 5. If desired, small holes or openings may be formed in the head portion 40 to receive projections from a driver. These small holes or openings may have a circular configuration and be offset from the slits which separate the resiliently deflectable sections or arms 48.

In the embodiment of the invention illustrated in FIGS. 1-6, the end surfaces 78 on the resiliently deflectable sections or arms 48 have the same radius of curvature as the side surface 74 of the recess 70. However, the end surfaces 78 on the resiliently deflectable sections or arms 48 may have a radius of curvature which is either greater or less than the radius of curvature of the side surface 74 of the recess 70. The illustrated end surfaces 78 have a convex configuration. However, the end surfaces 78 may have a concave configuration to provide corners which would tend to indent the side surface 74 of the recess 70 as the bone screw 24 is tightened.

Each of the resiliently deflectable sections or arms 48 has parallel side surfaces 64 and 90. The side surfaces 64 and 90 (FIG. 4) are disposed in planes which extend parallel to the longitudinal central axis of the shank portion 42. The side surfaces 64 and 90 are offset to opposite sides of the central axis of the shank portion 42 and do not intersect the central axis of the shank portion 42. When the bone screw 24 is in the installed condition of FIGS. 5 and 6, the side surfaces 64 and 90 extend perpendicular to the longitudinal central axis of the shank portion 42.

Although the side surfaces 64 and 90 on any one of the resiliently deflectable sections or arms 48 are parallel, the side surfaces may be skewed relative to each other. For example, the side surfaces 64 and 90 may flare outward from each other. If this is done, the arcuate extent of the outer end surfaces 78 (FIG. 4) would be greater than the arcuate extent of the intersections between the resiliently deflectable sections or arms 48 and the central portion 46 of the head portion 40. This would increase the area of contact between the outer end surfaces 68 and the side surface 74 of the recess 70 (FIG. 6).

As another example of an optional spatial relationship between the side surfaces 64 and 90 of the resiliently deflectable sections or arms 48, the side surfaces may taper inward toward each other. If this is done, the arcuate extent of the outer end surfaces 78 (FIG. 4) would be less than the arcuate extent of the intersections between the resiliently deflectable sections or arms 48 and the central portion 46 of the head portion 40.

If desired, one or more teeth may be formed in the radially outer ends of the resiliently deflectable sections or arms 48. These teeth may be formed in the outer end surfaces 78. Mating teeth may be formed in the side surface 74 of the recess 70. This would result in the teeth in the outer ends of the resiliently deflectable sections or arms 48 moving along the teeth in the side surface 74 of the recess 70 with a ratchet action as the bone screw 24 is moved to the installed condition of FIGS. 5 and 6.

Based on the foregoing description, it is apparent that the present invention provides a new and improved method and apparatus for use in association with bone 10 in a patient's body. The apparatus includes a plate 26 which is secured to the bone 10 in the patient's body by an improved bone screw 24. When they are installed in the patient's body, the plate 26 and the bone screw 24 cooperate to retain the bone screw against loosening. The installed bone screw 24 (FIG. 5) has an outer side surface 36 which is aligned with an outer side surface 32 of the plate to minimize projections from the plate.

The present invention has a plurality of different features. These features may be utilized together in the manner disclosed herein. Alternatively, the various features of the invention may be utilized separately and/or in combination with the features from the prior art.

From the above description of the invention, those skilled in the art will perceive improvements, changes and modifications which may be made in the specific embodiments of the invention illustrated herein. Such improvements, changes and modifications within the skill of the art are intended to be cover by the claims.

Having described the invention, the following is claimed:

1. A method of securing a plate to a bone in a patient's body, said method comprising the steps of:
positioning the plate adjacent to the bone in the patient's body,
moving a shank portion of a bone screw through an opening in a bottom of a recess in the plate into engagement with the bone in the patient's body,
rotating the bone screw about a longitudinal central axis of the shank portion to move an external thread convolution on the shank portion into the bone in the patient's body,
sliding end portions of resiliently deflectable sections of a head portion of the bone screw along the bottom of the recess during rotation of the bone screw about a longitudinal central axis of the shank portion,
deflecting the resiliently deflectable sections of the head portion in a direction away from a leading end of the shank portion under the influence of force transmitted from the bottom of the recess to the deflectable sections of the head portion as the bone screw is rotated about the longitudinal central axis of the shank portion, said step of deflecting the resiliently deflectable sections of the head portion includes moving outer end surfaces on the resiliently deflectable sections of the head portion outward from the longitudinal central axis of the shank portion into engagement with a side surface of the recess, and
retaining the bone screw against rotation relative to the plate under the influence of force transmitted between the outer end surfaces on the resiliently deflectable sections of the head portion and the side surface of the recess.

2. A method as set forth in claim 1 wherein said step of rotating the bone screw about the longitudinal central axis of the shank portion includes applying force against side surfaces of the resiliently deflectable portions at locations disposed between the outer end surfaces on the resiliently deflectable sections of the head portion and a central portion of the head portion.

3. A method as set forth in claim 1 wherein said step of deflecting the resiliently deflectable sections of the head portion includes moving side surfaces which are disposed on the resiliently deflectable sections of the head portion into alignment with a side surface of the plate which faces away from the bone in the patient's body.

4. An apparatus for use in association with bone in a patient's body, said apparatus comprising:
a plate having first and second major side surfaces, said plate at least partially defining a recess extending from said first major side surface toward said second major side surface, said recess having a bottom surface which is spaced from said second major side surface, said recess has a side surface which extends transverse to said first major side surface of said plate and to said bottom surface of said recess, said plate having an opening which extends from said bottom surface of said recess to said second major side surface of said plate, and
a bone screw having a head portion and a shank portion, said shank portion is integrally formed as one piece with said head portion and extends from said head portion to a leading end portion of said shank portion, said shank portion having an external thread convolution which is engagable with the bone in the patient's body to secure said plate to the bone with said shank portion extending through the opening in said plate and with said head portion engaging said bottom surface of said recess, said head portion having a plurality of resiliently deflectable sections which extend outward from a central portion of said head portion in a direction toward said leading end of said shank portion when said bone screw is in an initial condition, each of said resiliently deflectable sections having an end portion which is disposed radially outward from a portion of said shank portion and which is spaced from said head portion in the direction toward said leading end of said shank portion when said bone screw is in the initial condition, said bone screw being rotatable about a longitudinal central axis of said shank portion to turn said external thread convolution on said shank portion into the bone in the patient's body, said resiliently deflectable sections of said head portion move into sliding engagement with said bottom surface of said recess and are deflected in a direction away from the leading end of said shank portion as said bone screw is rotated about the longitudinal central axis of said shank portion, said end portions of said sections of said head portion move into engagement with said side surface of said recess as said sections of said head portion are deflected to retain said bone screw against rotation relative to said plate.

5. An apparatus as set forth in claim 4 wherein side surfaces on said central portion and on said resiliently deflectable sections are aligned with said first major side surface of said plate when said bone screw has been tightened by rotation about the longitudinal central axis of said bone screw.

6. An apparatus as set forth in claim 4 wherein said end portions of said resiliently deflectable sections of said head portion have arcuate end surfaces which have radii of curvature equal to a radius of curvature of said side surface of said recess.

7. An apparatus as set forth in claim 4 wherein each of said resiliently deflectable sections has a drive surface which is engaged by a drive member to apply force to said bone screw to rotate said bone screw relative to said plate.
